(19) United States
(12) Patent Application Publication
(10) Pub. No.: US 2008/0152946 A1
(43) Pub. Date: Jun. 26, 2008

(54) Novel fluorene compounds, hole injection materials/hole transport materials containing said fluorene compounds, a light emitting element containing said fluorene compounds and methods of preparation thereof

(76) Inventors: Feng-Wen Yen, Hsin-Chu (TW); Chao-you Chiu, Hsin-Chu (TW)

Correspondence Address: VENABLE LLP, P.O. BOX 34385, WASHINGTON, DC, 20043-9998, US

(21) Appl. No.: 11/643,821

(22) Filed: Dec. 22, 2006

(51) Int. Cl. H01J 1/62; B05D 5/12; C07C 211/00

(52) U.S. Cl. 428/690; 427/67; 564/428

(57) ABSTRACT

Novel fluorene compounds having the following formula are disclosed:

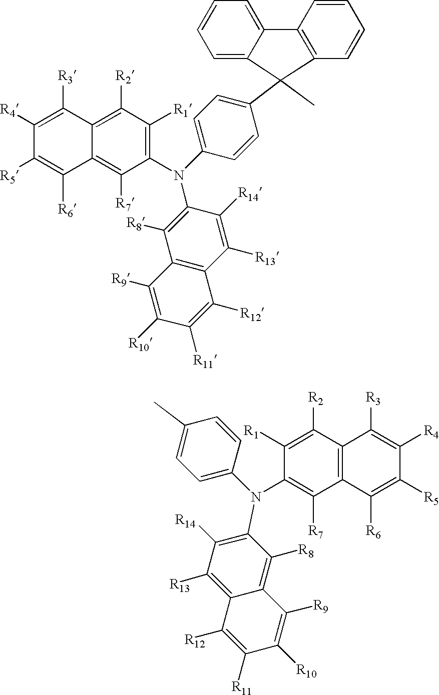

wherein R1-R14 and R1′-R14′ are respectively H, halogen, C1-C6 alkyl, alkyloxyl or cycloalkyl. The present also discloses their method of preparation and their usage as the hole injection material or the hole transport material of an organic light emitting diode, and the light emitting diode containing the invented fluorene compounds and its preparation method.

### FIELD OF THE INVENTION

The present invention relates to novel fluorene compounds, hole injection materials/hole transport materials containing the fluorene compounds, a light emitting element containing the fluorene compounds and their preparation methods.

### BACKGROUNDS OF THE INVENTION

Although the industrial development of the organic light emitting diodes starts about two decays ago, its applications have caught the attentions of the industry widely in recent years. The most popular applications of the organic light emitting diodes are in the displaying devices. Researchers in the industry and in the research institutes are devoted in the development of light emitting materials of higher yields and longer lifetimes.

The basic structure of the organic light emitting diodes contains three layers. They are: Two electrode layers and an organic light emission layer positioned between them. In the two electrodes, the one in connection with the negative pole of the power supply is the electron injection layer, which generates electrons when a voltage is applied. The electrode in connection with the positive pole of the power supply is the hole injection layer, which generates holes when a voltage is applied. When the electrons and the holes meet in the organic light emitting layer, light is generated. Based on the basic structure as described above, an electron transport layer may be added between the electron injection layer and the light emitting layer and a hole transport layer may be added between the hole injection layer and the light emitting layer.

U.S. Pat. No. 6,586,120, issued to Tao et al., discloses an “electro luminescent device comprising fluorene compounds.” In this US patent fluorene compounds having the following formula are disclosed:

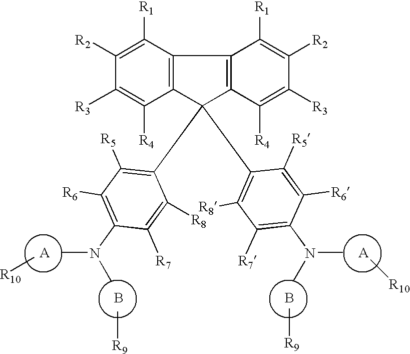

wherein R1-R4, R5-R8, and R5′-R8′ are respectively H, halogen, CN, NO2, amino, alkylamino, arylamino, dialkylamino, diarylamino, hydroxyl, alkoxyl, aryloxyl, heteroaryloxyl, mercapto, alkylthio, arylthio, heteroarylthio, alkyl, aryl, heteroaryl, or heterocyclyl; A and B are respectively phenyl, naphthyl, or phenanthryl; and R9 and R10 are respectively H, aryl, heteroaryl, aryloxyl, or heteroaryloxyl; provided that if both A and B are phenyl, one of R9 and R10 is not H.

Although the above US patent includes a wide variety of compounds in its claims, only one compound was disclosed in the embodiments of the patent specification. The compound as disclosed is: 9,9-bis{4-[di-(p-biphenyl)aminophynyl]}fluorene (BPAPF).

Nevertheless, although the fluorene compound disclosed in the above-mentioned US patent provides greater luminance, it has a relatively short lifetime, which is insufficient for the commercial applications. At the same time, its yields are relatively low due to its relatively high operation voltages. Generally speaking, the fine flatness of ITO substrates are desirable for the manufacturers of the display panel. Defects in the flatness of the substrate would lead to the leakage of electricity in the display panel, after the hole transport layer is deposited onto the substrate. The leakages would impact the transportation of the holes from the ITO substrate to the light emission layer. In the preparation of the light emitting element, applying a hole injection layer on the ITO layer as the buffer layer would improve the flatness of the substrate and make the lifetime of the light emission element longer. However, applying the hole injection layer would make the operation voltage of the element going higher and decrease its luminance, whereby damaging the yields of the light emitting element.

It is thus necessary to provide a new material for the hole injection layer or the hole transport layer of the organic light emitting diode (OLED), whereby the yield of the OLED may be increased.

It is also necessary to provide a new material for the hole injection layer or the hole transport layer of the OLED, whereby the luminance of the OLED may be increased, without increasing the operation voltage of the device.

It is also necessary to provide a new material for the hole injection layer or the hole transport layer of the OLED whereby the OLEDs so prepared are stable and have longer lifetimes.

It is also necessary to provide a thermally stable material for the hole injection layer or the hole transport layer of the OLED.

### OBJECTIVES OF THE INVENTION

An objective of the present invention is to provide novel fluorene compounds that may be used in the OLED as the material of the hole injection layer or the hole transport layer.

Another objective of this invention is to provide a material for the hole injection layer or the hole transport layer of OLED whereby the luminance of the OLEDs so prepared is enhanced.

Another objective of this invention is to provide a new material for the hole injection layer or the hole transport layer of the OLED whereby the OLEDs so prepared are stable and have longer lifetimes.

Another objective of this invention is to provide a thermally stable material for the hole injection layer or the hole transport layer of the OLED.

Another objective of this invention is to provide a method for the preparation of the above-mentioned fluorene compounds.

Another objective of this invention is to provide a method for the preparation of the above-mentioned hole injection layer and hole transport layer for OLED.

Another objective of this invention is to provide a method for the preparation of OLED's wherein the invented fluorene compounds are used as the main material of the hole injection layer or the hole transport layer.

### SUMMARY OF THE INVENTION

According to the present invention, new fluorene compounds having the following formula are disclosed:

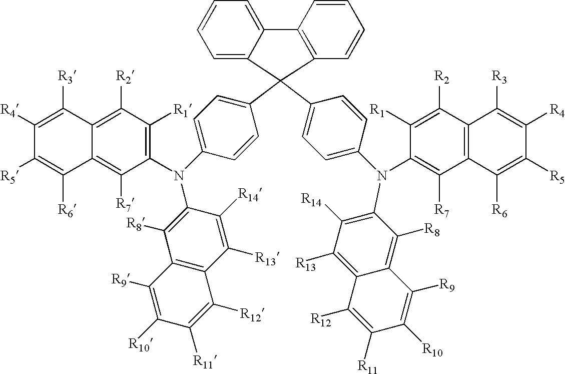

wherein R1-R14 and R1′-R14′ are respectively H, halogen, C1-C6 alkyl, alkyloxyl or cycloalkyl.

The present also discloses a method for the preparation of these fluorene compounds and their usage as materials for the hole injection layer or the hole transport layer of an organic light emitting diode (OLED). In addition, the present invention also discloses the OLED in which the invented fluorene compounds are used as the main material of the hole injection layer or the hole transport layer, and the method for the preparation of the OLED.

The fluorene compounds as disclosed in the present invention in particular comprises 9,9-Bis[4-d(naphtha-2-yl)aminophenyl]fluorene. When the invented fluorene compounds are used as the materials of the hole injection layer or the hole transport layer for the OLED, the OLED exhibits relatively high luminance without an increased operation voltage, longer lifetime and better thermal stability.

The invented fluorene compounds, the hole injection layer and the hole transport layer containing the invented fluorene compounds, the OLED containing the invented fluorene compounds and their methods for preparation will be described in the followings by illustrations. It shall be noted that the preferred embodiments as shown in the detailed description are for illustration purposes only. They shall not be used to limit the scope of protection of the present invention.

### BRIEF DESCRIPTION OF THE DRAWINGS

In the drawings, FIG. 1 shows the curves of the glass transformation temperature (Tg), the crystallization temperature (Tc) and the melting temperature (Tm) of the fluorene compounds of the present invention.

FIG. 2 shows the curve of the thermal degradation temperature of the invented fluorene compounds.

Table I shows the natures of the present invention and several known chemicals when used as the material of the hole injection layer/hole transport layer of the organic light emitting diode.

### DETAILED DESCRIPTION OF THE INVENTION

The fluorene compounds of the present invention have the following formula:

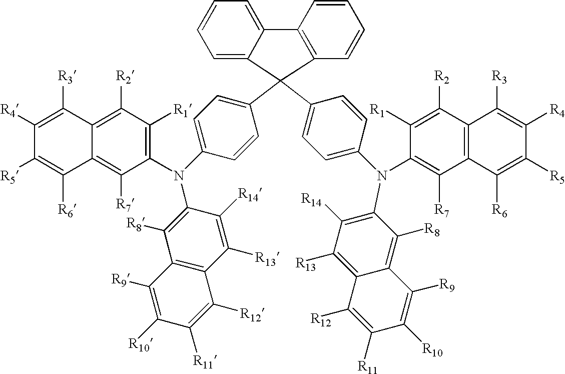

wherein R1-R14 and R1′-R14′ are respectively H, halogen, C1-C6 alkyl, alkyloxyl or cycloalkyl. The fluorene compounds are useful in the hole injection layer or the hole transport layer for the OLED.

The preparation of the invented fluorene compounds will be described first. 10 g 9,9-bis(4-aminobiphenyl)amine (0.029 mol), 36 g 2-bromonaphthalene (0.174 mol), 0.006 g palladium(II)acetate (0.00029 mol), 0.3 g 2-dicyclohexylphosphino biphenyl (0.00087 mol), 16.7 g sodium tert butoxide (0.174 mol) and 200 ml xylene are mixed and stirred in a 500 ml three-necked bottle and heated to 160° C. Reflux for 48 hours.

Use the “thin-layer chromatography” (EA:Hexane=1:3) to confirm that the reaction is complete. The produced liquid is thermo filtrated. The filtrate is collected and cooled. The filtrate is dropped into 1,000 ml acetic acid and stirred for 24 hours. The product is filtered and the residues are collected and crystallized in 10 times dichloromethane to obtain 6 g of the final product. The product is labeled as LT-N121.

The product is NMR-verified as 9,9-Bis[4-d(naphtha-2-yl)aminophenyl]fluorene, as follows: 500 Hz NMR (CDCl3) δ7.00 (d, J=9 Hz 4H), δ7.12 (d, J=9 Hz 4H), δ7.28 (d,d, J=9 Hz,2 Hz 4H), δ7.30-7.39 (m9, 12H), δ7.43 (d, J=2 Hz 4H), δ7.49 (d, J=7.5 Hz 2H), δ7.55 (d, J=8 Hz 4H). δ7.69 (d, J=8 Hz 4H), δ7.73 (d, J=7.5 Hz 4H), δ7.77 (d, J=7.5 Hz 2H).

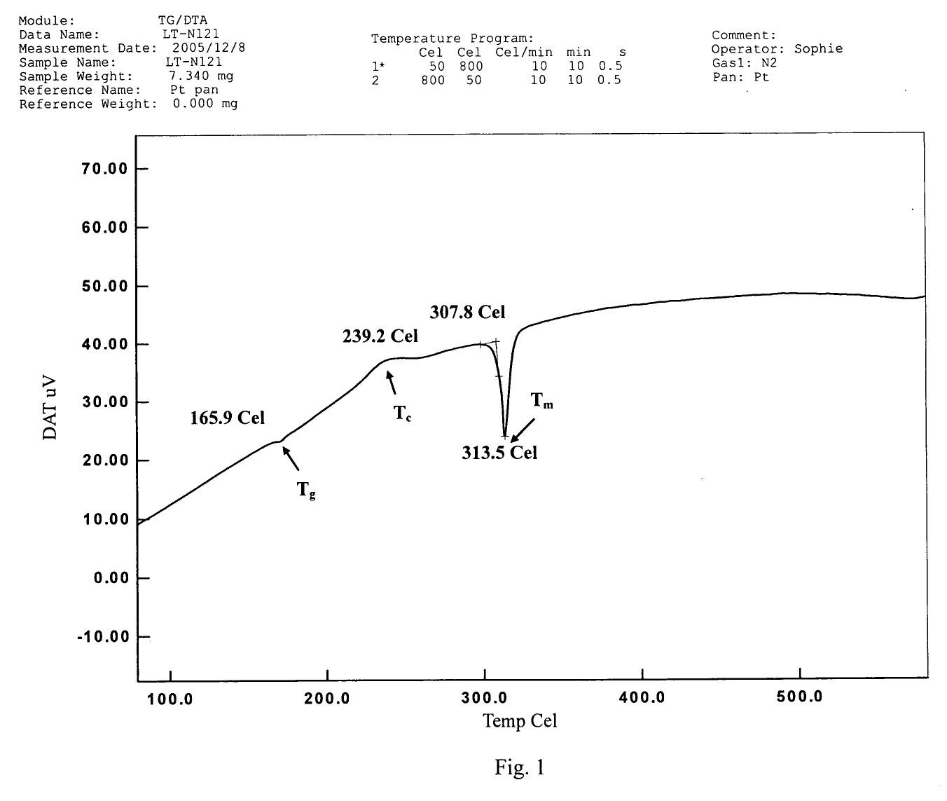

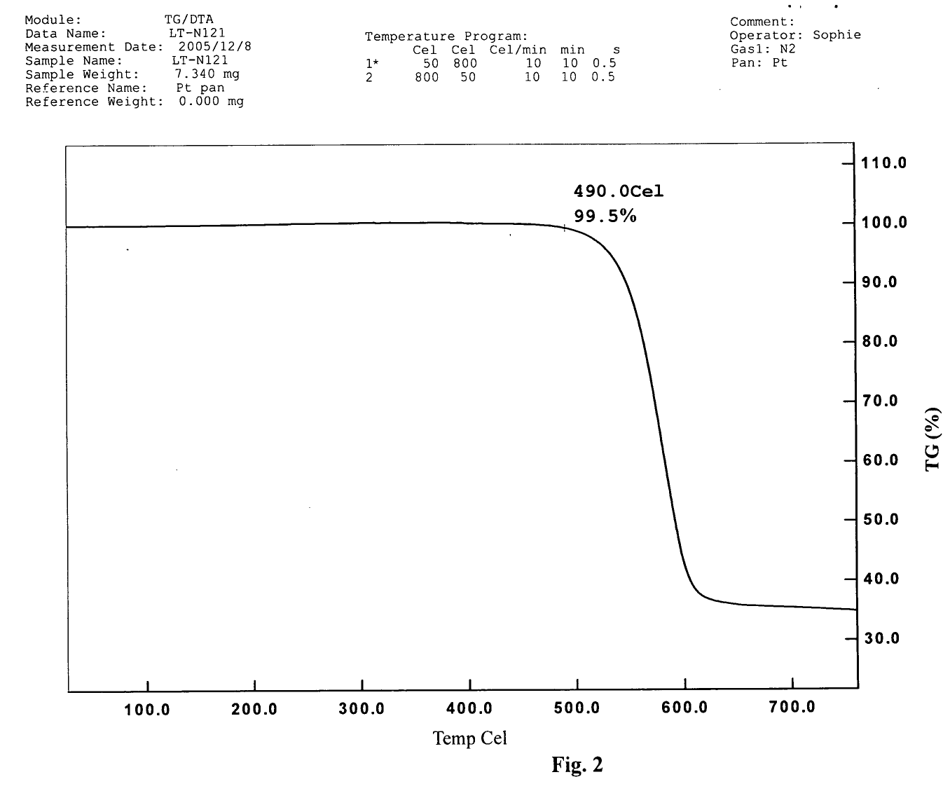

The physical property of the product is measured, including its glass transformation temperature (Tg), its crystallization temperature (Tc), melting temperature (Tm) etc. FIG. 1 shows the curves of the glass transformation temperature (Tg), the crystallization temperature (Tc) and the melting temperature (Tm) of the fluorene compounds of the present invention. As shown in the figure, the Tg, Tc and Tm of the invented fluorene compounds are 165.9° C., 239.2° C. and 307.8° C., respectively. The thermal degradation temperature of the product is measured in a thermogravimetric analyzer (TGA). The results are shown in FIG. 2. FIG. 2 shows the curve of the thermal degradation temperature of the invented fluorene compounds.

An organic light emitting diode (OLED) is prepared using the invented fluorene compounds and other known materials as the materials for the hole injection layer/hole transport layer of the OLED:

First electrode layer: A transparent electrode made of ITO, in the thickness of 150 nm.

Hole injection layer: The hole injection layer is prepared using the fluorene compound of this invention (LT-N121), the fluorene compound of the U.S. Pat. No. 6,586,120 (BPAPF), the LG-HIL product in the name of LG101 (LG-HIL), the Merck-HIL product in the name of TMM016 (Merck-HIL), 4,4′,4″-tris(N-3-methylphenyl-N-phenyl-amino)-triphenylamine (m-MTDATA) and the well-known N,N′-bis(naphthalene-1-yl)-N,N′-bis-(phenyl)-benzidine (NPB) as the main material, respectively. The hole injection layer is vapor-deposited on the first electrode layer, in the thickness as shown in Table I.

Hole transport layer: The hole transport layer is prepared using the invented fluorene compounds (LT-N121), the known BPAPF and NPB as the main material, as shown in Table I, and is deposited on the hole injection layer in the thickness as shown in Table I.

Light emission layer: 2% 4,4′-bis(9-ethyl-3-carbazovinylene)-1,1′-biphenyl (BCzVBi) doped in 4,4′-bis(2,2-diphenyl-ethen-1-yl)diphenyl (DPVBi) is prepared to form the blue light emission layer and is vacuum vapor-deposited on the hole transport layer, in the thickness of 30 nm.

Electron transport layer: The known tris-(8-hydroxyquinoline)aluminum (Alq₃) is prepared and deposited on the light emission layer, in the thickness of 20 nm.

Electron injection layer: Suited materials for the electron injection layer include LiF, MgO, Li₂O etc. In these embodiments the electron injection layer is prepared from LiF and is deposited on the electron transport layer, in the thickness of 0.5 nm.

Second electrode layer: Suited materials for the electrode layer include Al, Mg, Ca, Li, K etc. In the embodiments of this invention, Al is used as the material of the electrode layer and is deposited on the electron injection layer, in the thickness of 120 nm.

The light emitting elements are prepared as described above and are divided into die. The devices are connected with the power supply to measure their operation voltages, luminance, yields and lifetime. The results are shown in Table I. Table I shows the natures of the present invention and several known chemicals when used as the material of the hole injection layer/hole transport layer of the organic light emitting diode. In the table, the lifetimes are tested in the initial luminance of 500 cd/m², under the current density of 8-10 mA/cm².

TABLE I

| | | | Voltage | Luminance | Yield | Life-Time |
|---|---|---|---|---|---|---|
| | HIL(nm) | HTL(nm) | (V) | (cd/m²) | (cd/A) | (t_{1/2}, hr)* |
| Example 1 | LT-N121 | NPB | 6.5 | 528 | 7.23 | 800 |
| | (60 nm) | (10 nm) | | | | |
| Example 2 | LT-N121 | NPB | 5.5 | 413 | 6.58 | 780 |
| | (40 nm) | (10 nm) | | | | |
| Example 3 | LT-N121 | LT-N121 | 6.0 | 441 | 8.82 | 700 |
| | (60 nm) | (10 nm) | | | | |
| Comparable | BPAPF | BPAPF | 7.0 | 540 | 5.56 | 350 |
| Example 1 | (60 nm) | (10 nm) | | | | |
| Comparable | LG-HIL | NPB | 4.5 | 340 | 3.56 | 850 |
| Example 2 | (60 nm) | (10 nm) | | | | |
| Comparable | Merck-HIL | NPB | 5.5 | 339 | 5.21 | 500 |
| Example 3 | (60 nm) | (10 nm) | | | | |
| Comparable | m-TDATA | NPB | 7.0 | 477 | 5.66 | 100 |
| Example 4 | (60 nm) | (10 nm) | | | | |
| Comparable | NPB | NPB | 4.5 | 403 | 4.91 | 120 |
| Example 5 | (40 nm) | (10 nm) | | | | |
| Comparable | NPB | NPB | 5.0 | 444 | 4.77 | 180 |
| Example 6 | (60 nm) | (10 nm) | | | | |

*Initiate Luminance = 500 cd/m²
**Device be tested under current density = 8~10 mA/cm²

As shown in Table I, the fluorene compounds of the present invention provide the enhanced yields without increasing the operation voltage of the OLED. Their lifetime is several times that of the NPB. They exhibit higher Tg and perform excellent thermal stability. When they are used as the hole transport layer, the yields and the lifetime of the diode are both better than that of the BPABF.

As the present invention has been shown and described with reference to preferred embodiments thereof, those skilled in the art will recognize that the above and other changes may be made therein without departing from the spirit and scope of the invention.

What is claimed is:

1. Fluorene compounds having the formula of: 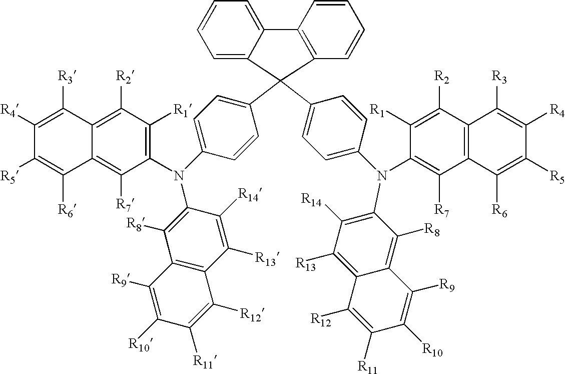
wherein R1-R14 and R1′-R14′ are respectively H, halogen, C1-C6 alkyl, alkyloxyl or cycloalkyl.

2. 9,9-Bis[4-d(naphtha-2-yl)aminophenyl]fluorene.

3. A hole injection layer for the organic light emission device, comprising a fluorene compound having the following formula as its main material: 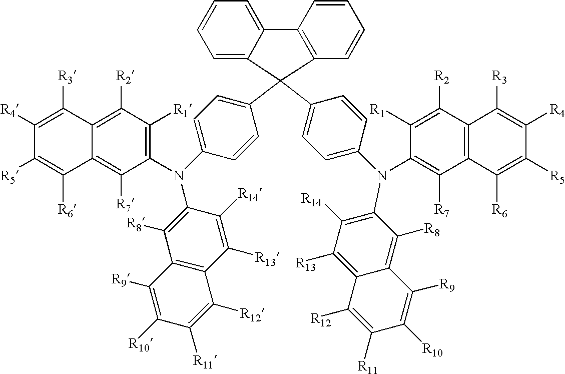
wherein R1-R14 and R1′-R14′ are respectively H, halogen, C1-C6 alkyl, alkyloxyl or cycloalkyl.

4. The hole injection layer according to claim 3, wherein said fluorene compound comprises 9,9-Bis[4-d(naphtha-2-yl)aminophenyl]fluorene.

5. A hole transport layer for the organic light emission device, comprising a fluorene compound having the following formula as its main material: 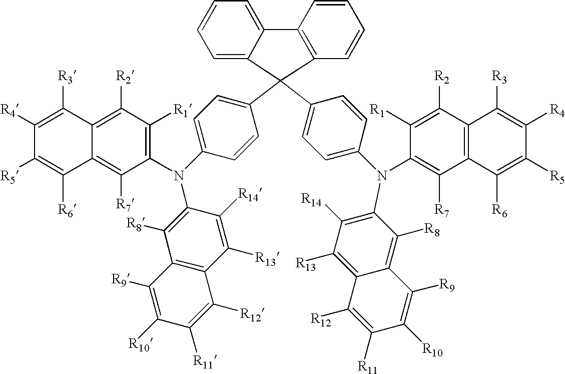
wherein R1-R14 and R1′-R14′ are respectively H, halogen, C1-C6 alkyl, alkyloxyl or cycloalkyl.

6. The hole transport layer according to claim 5, wherein said fluorene compound comprises 9,9-Bis[4-d(naphtha-2-yl)aminophenyl]fluorene.

7. A light emission device, comprising at least a first electrode layer, a hole injection layer, an electron injection layer and a second electrode layer; wherein said hole injection layer comprises as main material a fluorene compound having the following formula: 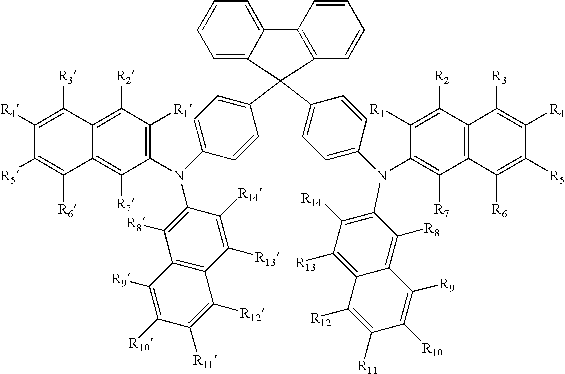
wherein R1-R14 and R1′-R14′ are respectively H, halogen, C1-C6 alkyl, alkyloxyl or cycloalkyl.

8. The light emission device according to claim 7, wherein said fluorene compound comprises 9,9-Bis[4-d(naphtha-2-yl)aminophenyl]fluorene.

9. The light emission device according to claim 7, further comprising a light emission layer positioned between said hole injection layer and said electron injection layer.

10. The light emission device according to claim 7, further comprising a hole transport layer positioned between said hole injection layer and said first electrode layer.

11. The light emission device according to claim 9, further comprising a hole transport layer positioned between said hole injection layer and said first electrode layer.

12. Method for the preparation of a fluorene compound having the following formula: 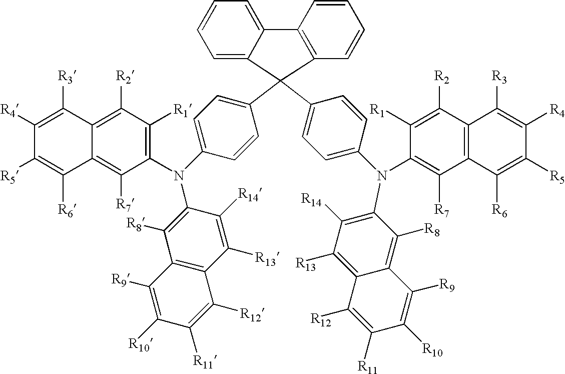
wherein R1-R14 and R1′-R14′ are respectively H, halogen, C1-C6 alkyl, alkyloxyl or cycloalkyl; said method comprising the steps of:
mixing by stirring and heating of a 9,9-bis(aminophenyl)amine compound and a naphthalene derivative under the existence of catalysts to obtain a product;
purifying said product; and
obtaining said fluorene compound.

13. The method according to claim 12, wherein said fluorene compound comprises 9,9-Bis[4-d(naphtha-2-yl)aminophenyl]fluorene.

14. Method for the preparation of an organic light emitting device, comprising the steps of:
preparation of a first electrode layer;
forming on said first electrode layer a hole injection layer;
forming on said hole injection layer an electron injection layer; and
forming on said electron injection layer a second electrode layer; wherein said hole injection layer comprises as main material a fluorene compound having the following formula: 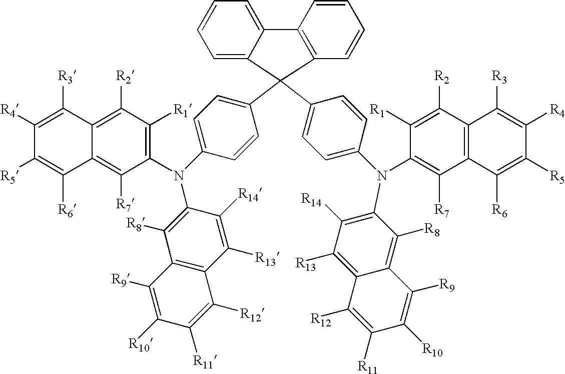
wherein R1-R14 and R1′-R14′ are respectively H, halogen, C1-C6 alkyl, alkyloxyl or cycloalkyl.

15. The method according to claim 14, wherein said fluorene compound comprises 9,9-Bis[4-d(naphtha-2-yl)aminophenyl]fluorene.

16. The method according to claim 14, further comprising the step of forming on said hole injection layer a light emission layer before said forming of said electron injection layer.

17. The method according to claim 14, further comprising the step of forming on said first electrode layer a hole transport layer before said forming of said hole injection layer.

18. The method according to claim 16, further comprising the step of forming on said first electrode layer a hole transport layer before said forming of said hole injection layer.
